(19) United States
(12) Patent Application Publication
(10) Pub. No.: US 2026/0227719 A1
(43) Pub. Date: Aug. 6, 2026

(54) ELECTROPHOTOGRAPHIC PHOTOSENSITIVE MEMBER, PROCESS CARTRIDGE, AND ELECTROPHOTOGRAPHIC APPARATUS

(71) Applicant: CANON KABUSHIKI KAISHA, Tokyo (JP)

(72) Inventors: NAO HIGUCHI, Shizuoka (JP); TAKEHIKO AOYAMA, Shizuoka (JP); AKIRA SAKAKIBARA, Shizuoka (JP)

(21) Appl. No.: 19/450,622

(22) Filed: Jan. 15, 2026

(30) Foreign Application Priority Data
Jan. 31, 2025 (JP) 2025-014459

(51) Int. Cl. G03G 5/06; C07C 211/54

(52) U.S. Cl. CPC G03G 5/061446; C07C 211/54; G03G 2215/00957; G03G 2215/00962; G03G 2221/183

(57) ABSTRACT

An electrophotographic photosensitive member includes a support, a charge generation layer on the support, and a charge transport layer on the charge generation layer, the charge transport layer constituting a surface layer. The charge transport layer includes a charge transport material, methyl palmitate, and a binder resin. The charge transport material includes a compound having a specified structure.

### BACKGROUND

### Field of the Technology

The present disclosure relates to an electrophotographic photosensitive member, and a process cartridge and an electrophotographic apparatus that include the electrophotographic photosensitive member.

### Description of the Related Art

In the field of the electrophotographic apparatus in recent years, from the viewpoint of decreasing the maintenance frequency and improving the usability, there has been demand for an image forming apparatus that can stably output high-quality images with a higher page yield of the copier main body or the cartridge. In order for an electrophotographic apparatus to achieve a higher page yield and a higher printing speed, it is necessary to address electrophotographic photosensitive member-associated adverse effects that occur in images and become prominent in long-term use.

An electrophotographic image forming method involves a charging step, an exposing step, a developing step, a transferring step, and a cleaning step. In recent years, image forming methods having no cleaning step have also been available. In an electrophotographic apparatus, an electrophotographic photosensitive member is in contact with a contact member such as an intermediate transfer belt at an appropriate relative rotation speed.

In a typical image forming method, corona products occur as a result of discharging in the charging step. When corona products remain on the surface of the electrophotographic photosensitive member, the corona products absorb water in air and the surface resistance of the electrophotographic photosensitive member decreases. When images are formed by using the electrophotographic photosensitive member in such a state, carrier migration is induced on the surface of the electrophotographic photosensitive member, and the intended electrostatic latent image becomes disarrayed. As a result, an adverse effect known as “image smearing” occurs in the image, in which a somewhat blurred image is output. Image smearing is an adverse effect in an image affected by the amount of accumulated corona products and the amount of water in air; thus, image smearing tends to occur prominently when the electrophotographic photosensitive member is used for a long time in a high-temperature, high-humidity environment.

This disadvantage is likely to arise in electrophotographic apparatuses that are required to have longer lifetime than typical apparatuses. Examples of the measures that address this disadvantage include improving the surface of the electrophotographic photosensitive member and the resin material to suppress accumulation of corona products on the electrophotographic photosensitive member surfaces and installing, into an image forming apparatus, a mechanism that removes corona products that have accumulated on the electrophotographic photosensitive member surface. In order to implement these measures, for example, following techniques have been proposed.

Japanese Patent Laid-Open No. 2017-138458 proposes a technique of suppressing accumulation of corona products by placing high-purity silicon particles on the surface of the electrophotographic photosensitive member.

Japanese Patent Laid-Open No. 2009-15306 proposes a technique of suppressing accumulation of corona products by enhancing discharge immunity by using, as the surface layer of the electrophotographic photosensitive member, a cured material having a specific structure and a charge transport function.

Japanese Patent Laid-Open No. 2001-305764 proposes an electrophotographic photosensitive member with excellent sensitivity, the electrophotographic photosensitive member using a diamine derivative as a charge transport material.

### SUMMARY

However, in an image forming apparatus required to stably output high-quality images in the long-term use, it has not been possible, according to Japanese Patent Laid-Open No. 2017-138458, to maintain an appropriate surface condition due to detachment of particles. Especially in recent years, the toner particles have become increasingly smaller in diameter in order to form high-quality images. In order to prevent faulty cleaning caused by small-diameter toner particles, the contact pressure and the contact area of the cleaning blade have increased from what have been before, and this more or less induces detachment of particles from the electrophotographic photosensitive member surface.

In Japanese Patent Laid-Open No. 2009-15306, the image density changed in the long-term use, and high-quality images could not be output. This is presumably due to the degradation of the carrier transport function caused by the long-term use. In order to stably output high-quality images in long-term use, the carrier transport material is required to have the endurance and the functional stability; thus, an appropriate carrier transport material needs to be selected for an appropriate layer.

In Japanese Patent Laid-Open No. 2001-305764, image smearing occurred in long-term use in a high-temperature, high-humidity environment. The cause for this is presumably that the carrier transport material having a molecular weight larger than those of typical materials is prone to remain on the electrophotographic photosensitive member surface and thus induces accumulation of corona products.

The present disclosure provides an electrophotographic photosensitive member that can withstand long-term use in a high-temperature, high-humidity environment while suppressing image smearing.

This can be achieved through the present disclosure below. That is, an electrophotographic photosensitive member according to an aspect of the present disclosure is an electrophotographic photosensitive member that includes a support, a charge generation layer on the support, and a charge transport layer on the charge generation layer, the charge transport layer constituting a surface layer, in which the charge transport layer includes a charge transport material, methyl palmitate, and a binder resin, and the charge transport material includes a compound having a structure represented by formula (1):

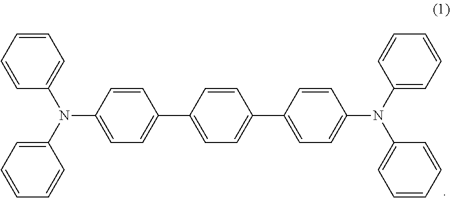 [SMILES: c1ccc(N(c2ccccc2)c2ccc(-c3ccc(-c4ccc(N(c5ccccc5)c5ccccc5)cc4)cc3)cc2)cc1]

A process cartridge according to another aspect of the present disclosure is a process cartridge detachably attachable to a main body of an electrophotographic apparatus, the process cartridge integrally supporting and including the electrophotographic photosensitive member described above, and at least one device selected from the group consisting of a charging device, a developing device, and a cleaning device.

An electrophotographic apparatus according to yet another aspect of the present disclosure is an electrophotographic apparatus that includes the electrophotographic photosensitive member described above, a charging device, an exposing device, a developing device, and a transferring device.

Features of the present disclosure will become apparent from the following description of embodiments with reference to the attached drawings. The following description of embodiments is described by way of example.

### BRIEF DESCRIPTION OF THE DRAWINGS

FIG. 1 illustrates an example of a layer structure of an electrophotographic photosensitive member according to the present disclosure.

FIG. 2 is a diagram illustrating an example of a schematic structure of an electrophotographic apparatus that includes process cartridges equipped with electrophotographic photosensitive members and charging devices.

### DESCRIPTION OF THE EMBODIMENTS

Embodiments of the technology of the present disclosure will now be described. Electrophotographic photosensitive member

An electrophotographic photosensitive member of the present disclosure includes a support, a charge generation layer on the support, and a charge transport layer on the charge generation layer. The electrophotographic photosensitive member according to the present disclosure can be used as a cylindrical electrophotographic photosensitive member in which a charge generation layer and a charge transport layer are sequentially formed on a cylindrical support; alternatively, the electrophotographic photosensitive member can assume a belt form or a sheet form.

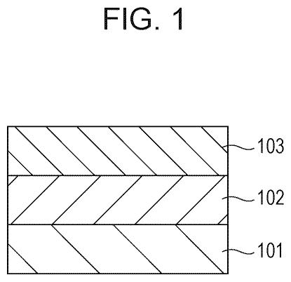

FIG. 1 is a diagram illustrating an example of a layer structure of the electrophotographic photosensitive member. In FIG. 1, 101 denotes a support, 102 denotes a charge generation layer, and 103 denotes a charge transport layer of the present disclosure.

The electrophotographic photosensitive member of the present disclosure can be used in an image forming method that includes a charging step of charging a surface of an electrophotographic photosensitive member; an exposing step of exposing the charged electrophotographic photosensitive member to form an electrostatic latent image; a developing step of supplying a toner to the electrophotographic photosensitive member having the electrostatic latent image formed thereon so as to form a toner image; a transferring step of transferring the toner image formed on the electrophotographic photosensitive member; and a cleaning step of removing the toner remaining on the electrophotographic photosensitive member in the transferring step.

Alternatively, the electrophotographic photosensitive member of the present disclosure can be used not only in the aforementioned image forming method but also in an image forming method that lacks the cleaning step of the aforementioned image forming method.

An example of the method for producing the electrophotographic photosensitive member of the present disclosure is a method that involves preparing coating solutions for respective layers described below, applying the coating solutions in the desired order of layers, and drying the applied coating solutions. Here, examples of the method for applying the coating solution include dip coating, spray coating, inkjet coating, roll coating, die coating, blade coating, curtain coating, wire bar coating, and ring coating. Among these, dip coating can be employed from the viewpoint of efficiency and productivity.

An electrophotographic photosensitive member according to the present disclosure includes a charge generation layer disposed on a conductive substrate and a charge transport layer disposed on the charge generation layer and constituting a surface layer, and features that the charge transport layer contains a charge transport material, methyl palmitate, and a binder resin and that the charge transport material contains a compound having a structure represented by formula (1).

Due to these features, the electrophotographic photosensitive member can be used longer compared to the related art and can stably output high-quality images in long-term use at high temperature and high humidity. The mechanism by which the features of the present disclosure address the disadvantages is presumably as follows.

The cause of the image smearing is the moisture in air and the accumulated corona products on the electrophotographic photosensitive member surface.

When accumulated corona products on the surface of the electrophotographic photosensitive member absorb moisture in air, the surface resistance of the electrophotographic photosensitive member decreases. As a result, carrier migration on the electrophotographic photosensitive member surface is induced, and the intended electrostatic latent image becomes disarrayed. As a result, blurred images are output.

The inventors of the present disclosure have made the following assumptions. When a charge transport material containing a compound having a structure represented by formula (1) mentioned above is used in the charge transport layer constituting the surface layer of the electrophotographic photosensitive member, a good charge transport function is exhibited over a long period of time, and high-quality images can be stably output in the long-term use at high temperature and high humidity. However, the charge transport material contained in the charge transport layer generates corona products when affected by discharging in the image forming step. These corona products are difficult to remove from the electrophotographic photosensitive member surface and accumulate on the electrophotographic photosensitive member surface in the long-term use, thereby causing the image smearing.

To address this, methyl palmitate is added to the charge transport layer constituting the surface layer to suppress occurrence of image smearing. Methyl palmitate also generates corona products as with the charge transport material described above, but shares the influence of discharging with the charge transport material. Thus, compared to the case in which methyl palmitate is not contained, the corona products generated when methyl palmitate is contained contain less corona products derived from the charge transport material and more corona products derived from methyl palmitate. In addition, the molecular weights of the generated corona products are dependent on the molecular weights of the original substances influenced by discharging. Compared to the molecular weight of the charge transport material, the molecular weight of methyl palmitate is smaller. In other words, the molecular weights of the corona products generated from these substances have a tendency in which the molecular weights of the corona products derived from methyl palmitate are smaller. Corona products with small molecular weights are easier to remove since the corona products tend to diffuse into air or come off from the electrophotographic photosensitive member surface when being rubbed with other members. As a result, the electrophotographic photosensitive member of the present disclosure having the aforementioned features can suppress accumulation of corona products in the long-term use.

This effect is achieved by using the charge transport material and methyl palmitate in combination.

For example, a substance having a smaller molecular weight than methyl palmitate tends to detach during the course of image formation and rarely remains on the electrophotographic photosensitive member surface. In such a case, the charge transport material is exposed to discharging, and corona products that are not easily detachable from the electrophotographic photosensitive member surface are generated and accumulate easily. Conversely, a substance having a larger molecular weight than methyl palmitate does not easily detach from the electrophotographic photosensitive member surface but generates corona products that also tend not to detach from the electrophotographic photosensitive member surface. As a result, corona products keep accumulating on the electrophotographic photosensitive member surface. Furthermore, methyl palmitate has compatibility with resins and does not obstruct the charge transport function.

In sum, by adding methyl palmitate to the charge transport layer while securing the electrical characteristics as the charge transport layer by using the charge transport material having a structure represented by formula (1), methyl palmitate instead of the charge transport material takes on the influence of discharging and generates methyl palmitate-derived corona products throughout the long-term use. These corona products readily come off from the electrophotographic photosensitive member surface during the course of image formation, and thus accumulation of the corona products is suppressed. As such, the longer use compared to related art becomes possible,, and high-quality images can be stably output in the long-term use at high temperature and high humidity.

Furthermore, the value (MP/MB) of the ratio of the mass (MP) of methyl palmitate to the mass (MB) of the binder resin in the charge transport layer may be 0.1 or less. In such a range, adverse effects and smearing in the images caused by bleeding that can result from addition of large quantities of methyl palmitate can be suppressed, and thus high-quality images can be stably output in the long-term use at high temperature and high humidity.

Furthermore, the value (MC/MB) of the ratio of the mass (MC) of the charge transport material to the mass (MB) of the binder resin in the charge transport layer may be 0.1 or more and 0.5 or less or may be 0.1 or more and 0.3 or less. Within this range, the amount of the charge transport material added can be suppressed while ensuring the electrical characteristics that can withstand the long-term use, and thus the endurance of the charge transport layer can be increased. This is because the surface of the electrophotographic photosensitive member is scraped less. The electrophotographic photosensitive member is rubbed with other members in the image forming step. When scraping of the electrophotographic photosensitive member surface by this rubbing can be suppressed, changes in electrical characteristics of the electrophotographic photosensitive member can be suppressed, and so can the adverse effect in the image. The endurance of the charge transport layer is derived from the binder resin, and the endurance of the charge transport layer has a tendency to increase with the decrease in the content of non-binding substances, such as the charge transport material.

Thus, decreasing the content of non-binding substances without degrading the electrical characteristics contributes to improving the endurance of the electrophotographic photosensitive member. In order to acquire the sensitivity appropriate for the electrophotographic photosensitive member, the value of ratio of the mass of the charge transport material to the mass of the binder resin may be 0.1 or more.

Furthermore, in order to increase the strength of the charge transport layer, the value of the ratio of the mass of the charge transport material to the mass of the binder resin may be 0.5 or less. As a result, endurance of the electrophotographic photosensitive member is enhanced.

Furthermore, the charge transport layer may contain a particle. In this manner, the strength of the charge transport layer increases, and the endurance of the electrophotographic photosensitive member can be enhanced. Examples of the particle are as follows: indium tin oxide particles, zinc oxide particles, titanium oxide particles, tin oxide particles, aluminum oxide particles, alumina particles, boron nitride particles, zirconium oxide particles, indium oxide particles, lanthanum oxide particles, antimony tin oxide particles, silica particles, fluorine resin particles, polystyrene resin particles, and polyethylene resin particles.

The charge transport layer may contain, as the particle, at least one selected from the group consisting of an indium tin oxide particle, a zinc oxide particle, and a titanium oxide particle. In this manner, the resistance of the charge transport layer can be decreased while increasing the strength of the charge transport layer, and thus the endurance of the electrophotographic photosensitive member can be enhanced without obstructing charge migration.

The number-average primary particle diameter of the particle contained in the charge transport layer may be 10 nm or more and 500 nm or less or may be 30 nm or more and 200 nm or less.

Within this range, the strength of the charge transport layer is increased without obstructing exposure of the photosensitive layer, and the endurance of the electrophotographic photosensitive member can be enhanced. When the number-average primary particle diameter of the particle is 10 nm or more, the aggregation force of the particle decreases, the particle is thus more evenly distributed within the charge transport layer, the endurance is likely to be exhibited more evenly, and the strength that can withstand the long-term use can be obtained. When the number-average primary particle diameter of the particle is 500 nm or less, exposure of the photosensitive layer is rarely obstructed, and the sensitivity of the electrophotographic photosensitive member is rarely degraded.

Furthermore, the value (MF/MB) of the ratio of the mass (MF) of the particle contained in the charge transport layer to the mass (MB) of the binder resin in the charge transport layer may be 0.05 or more and 0.4 or less or may be 0.1 or more and 0.3 or less. Within this range, the strength of the charge transport layer increases, and the endurance of the electrophotographic photosensitive member can be enhanced. In order to acquire the effect of enhancing the strength of the charge transport layer by adding the particle, the value of the ratio of the mass (MF) of the particle to the mass (MB) of the binder resin may be 0.05 or more. Furthermore, in order to prevent the particle from obstructing the exposure of the photosensitive layer and to suppress degradation of the sensitivity, the value of the ratio of the mass (MF) of the particle to the mass (MB) of the binder resin may be 0.4 or less.

In addition, the compound having a structure represented by formula (1) may be a compound represented by formula (2) below.

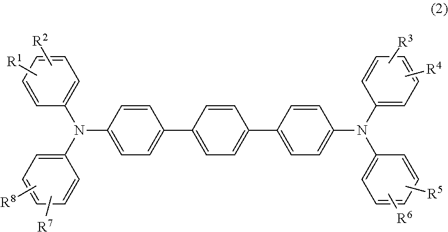

(In formula (2), R¹ to R⁸ each independently represent a hydrogen atom, a substituted or unsubstituted alkyl group, a substituted or unsubstituted alkylene group, a substituted or unsubstituted phenyl group, or a group represented by formula (2A) below.)

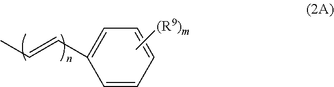

(In formula (2A), n represents an integer of 1 or more and 5 or less, m represents an integer of 0 or more and 5 or less, and in the case that m represents an integer of 2 or more, each R⁹ independently represents an alkyl group having 1 or more and 4 or less carbon atoms or an alkoxy group having 1 or more and 4 or less carbon atoms.)

In addition, R¹ to R⁸ in formula (2) may each independently represent an alkyl group having 1 or more and 3 or less carbon atoms. In particular, the charge transport material contained in the charge transport layer may contain at least one compound selected from the group consisting of compounds represented by formulae (3) to (5) below. In this manner, the electrical characteristics as the electrophotographic photosensitive member are further enhanced, and high-quality images can be stably output in the long-term use.

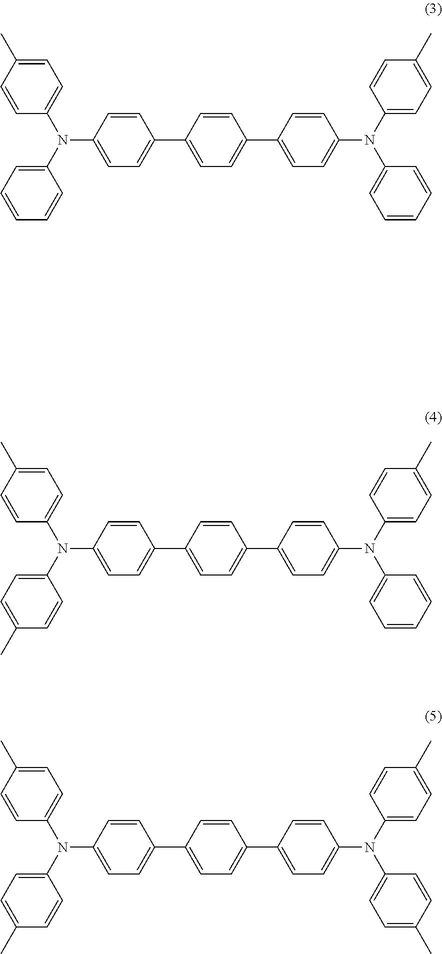 [SMILES: Cc1ccc(N(c2ccccc2)c2ccc(-c3ccc(-c4ccc(N(c5ccccc5)c5ccc(C)cc5)cc4)cc3)cc2)cc1 ; Cc1ccc(N(c2ccccc2)c2ccc(-c3ccc(-c4ccc(N(c5ccc(C)cc5)c5ccc(C)cc5)cc4)cc3)cc2)cc1 ; Cc1ccc(N(c2ccc(C)cc2)c2ccc(-c3ccc(-c4ccc(N(c5ccc(C)cc5)c5ccc(C)cc5)cc4)cc3)cc2)cc1]

Furthermore, the particle contained in the charge transport layer may be an indium tin oxide particle. In this manner, the resistance of the charge transport layer can be further decreased while increasing the strength of the charge transport layer, and thus the endurance of the electrophotographic photosensitive member can be enhanced without obstructing charge migration.

Note that this mechanism described thus far is based on the assumption, and this assumption does not affect the technical scope of the present disclosure in any way.

### Support

In the present disclosure, the electrophotographic photosensitive member may include a support. In the present disclosure, the support may be a conductive support having electroconductivity. Examples of the shape of the support include a cylindrical shape, a belt shape, and a sheet shape. In particular, the support may be a cylindrical support. The surface of the support may be subjected to an electrochemical treatment such as anodization, a blasting treatment, a cutting treatment, or the like.

The material for the support may be metal, resin, glass, or the like. Examples of the metal include aluminum, iron, nickel, copper, gold, stainless steel, and alloys thereof. In particular, the support may be an aluminum support containing aluminum.

The resin and the glass may be made electrically conductive by mixing a conductive material or providing a coating of a conductive material, for example.

If appropriate, an undercoat layer or the like may be provided on the support.

### Photosensitive Layer

The charge generation layer may contain a charge generation material and a resin.

Examples of the charge generation material include azo pigments, perylene pigments, polycyclic quinone pigments, indigo pigments, and phthalocyanine pigments. In particular, azo pigments and phthalocyanine pigments may be used. Of the phthalocyanine pigments, oxytitanium phthalocyanine pigments, chlorogallium phthalocyanine pigments, and hydroxygallium phthalocyanine pigments may be used.

The content of the charge generation material in the charge generation layer relative to the total mass of the charge generation layer may be 40 mass % or more and 85 mass % or less or may be 60 mass % or more and 80 mass % or less.

Examples of the resin include polyester resin, polycarbonate resin, polyvinyl acetal resin, polyvinyl butyral resin, acrylic resin, silicone resin, epoxy resin, melamine resin, polyurethane resin, phenolic resin, polyvinyl alcohol resin, cellulose resin, polystyrene resin, polyvinyl acetate resin, and polyvinyl chloride resin. Among these, polyvinyl butyral resin may be used.

The charge generation layer may further contain additives such as an antioxidant and an ultraviolet absorber.

Specific examples thereof include hindered phenol compounds, hindered amine compounds, sulfur compounds, phosphorus compounds, and benzophenone compounds.

The charge generation layer can be formed by preparing a charge generation layer-forming coating solution containing the aforementioned materials and a solvent and forming and drying the charge generation layer-forming coating solution on a support. Examples of the solvent used in the coating solution include alcohol solvents, sulfoxide solvents, ketone solvents, ether solvents, ester solvents, and aromatic hydrocarbon solvents.

The thickness of the charge generation layer may be 0.10 μm or more and 1.5 μm or less or may be 0.15 μm or more and 1.0 μm or less.

### Charge Transport Layer

The charge transport layer of the present disclosure includes a charge transport material, methyl palmitate, and a binder resin.

An example of the charge transport material is a material having a triarylamine structure. In particular, a triarylamine compound having a structure represented by formula (1) above may be contained. Examples of the compound having a structure represented by formula (1) include, in addition to compounds represented by formulae (3) to (5), the following compounds.

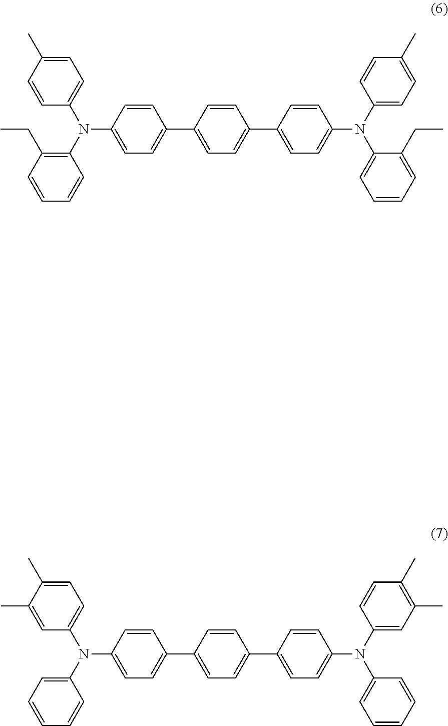

Examples of the binder resin include polyester resin, polycarbonate resin, acrylic resin, and polystyrene resin. Of these, polycarbonate resin and polyester resin may be used from the viewpoint of endurance. The polyester resin may be a polyarylate resin.

The charge transport layer may contain a particle for increasing the wear resistance. Specific examples thereof include indium tin oxide particles, zinc oxide particles, titanium oxide particles, tin oxide particles, aluminum oxide particles, alumina particles, boron nitride particles, zirconium oxide particles, indium oxide particles, lanthanum oxide particles, antimony tin oxide particles, silica particles, fluorine resin particles, polystyrene resin particles, and polyethylene resin particles. Among these, indium tin oxide particles, zinc oxide particles, or titanium oxide particles may be used from the viewpoint of sensitivity, and, in particular, indium tin oxide particles may be used.

The particle contained in the charge transport layer of the present disclosure may be surface-treated. An example of the surface treatment is a treatment that uses a hydrophobic treatment agent. Examples of the hydrophobic treatment agent include chlorosilanes, alkoxysilanes, silazanes, silicone oils, and siloxanes. Specific examples are as follows:

Chlorosilanes such as methyltrichlorosilane, dimethyldichlorosilane, phenyltrichlorosilane, diphenyldichlorosilane, t-trimethylchlorosilane, butyldimethylchlorosilane, and vinyltrichlorosilane;
alkoxy silanes such as tetramethoxysilane, methyltrimethoxysilane, dimethyldimethoxysilane, phenyltrimethoxysilane, diphenyldimethoxysilane, o-methylphenyltrimethoxysilane, p-methylphenyltrimethoxysilane, n-butyltrimethoxysilane, i-butyltrimethoxysilane, hexyltrimethoxysilane, octyltrimethoxysilane, decyltrimethoxysilane, dodecyltrimethoxysilane, tetraethoxysilane, methyltriethoxysilane, dimethyldiethoxysilane, phenyltriethoxysilane, diphenyldiethoxysilane, i-butyltriethoxysilane, decyltriethoxysilane, vinyltriethoxysilane, γ-methacryloxypropyltrimethoxysilane, γ-glycidoxypropyltrimethoxysilane, γ-glycidoxypropylmethyldimethoxysilane, γ-mercaptopropyltrimethoxysilane, γ-chloropropyltrimethoxysilane, γ-aminopropyltrimethoxysilane, γ-aminopropyltriethoxysilane, γ-(2-aminoethyl)aminopropyltrimethoxysilane, and γ-(2-aminoethyl)aminopropylmethyldimethoxysilane;
silazanes such as hexamethyldisilazane, hexaethyldisilazane, hexapropyldisilazane, hexabutyldisilazane, hexapentyldisilazane, hexahexyldisilazane, hexacyclohexyldisilazane, hexaphenyldisilazane, divinyltetramethyldisilazane, and dimethyltetravinyldisilazane;
silicone oils such as dimethyl silicone oil, methyl hydrogen silicone oil, methyl phenyl silicone oil, alkyl-modified silicone oil, chloroalkyl-modified silicone oil, chlorophenyl-modified silicone oil, fatty acid-modified silicone oil, polyether-modified silicone oil, alkoxy-modified silicone oil, carbinol-modified silicone oil, amino-modified silicone oil, fluorine-modified silicone oil, and terminally reactive silicone oil;
siloxanes such as hexamethylcyclotrisiloxane, octamethylcyclotetrasiloxane, decamethylcyclopentasiloxane, hexamethyldisiloxane, and octamethyltrisiloxane; and
fatty acids and metal salts thereof such as long-chain fatty acids such as undecylic acid, lauric acid, tridecylic acid, dodecylic acid, myristic acid, palmitic acid, pentadecylic acid, stearic acid, heptadecylic acid, arachidic acid, montanic acid, oleic acid, linoleic acid, and arachidonic acid, and salts of these fatty acids and metals such as zinc, iron, magnesium, aluminum, calcium, sodium, and lithium.

Of these, alkoxysilanes, silazanes, and silicone oils may be used to facilitate the hydrophobizing treatment. These hydrophobic treatment agents may be used alone or in combination.

The charge transport layer may further contain additives such as an antioxidants, an ultraviolet absorber, a plasticizer, a leveling agent, and a slip agent. Specific examples thereof include hindered phenol compounds, hindered amine compounds, sulfur compounds, phosphorus compounds, benzophenone compounds, siloxane-modified resin, and silicone oil.

The charge transport layer can be formed by preparing a charge transport layer-forming coating solution containing the aforementioned materials and a solvent and forming and drying the charge transport layer-forming coating solution on the charge generation layer. Examples of the solvent used in the coating solution include alcohol solvents, ketone solvents, ether solvents, ester solvents, aromatic hydrocarbon solvents, and amide solvents.

Specific examples are as follows:

Methanol, ethanol, isopropanol, butanol, octanol, acetone, cyclohexanone, ethylene glycol monomethyl ether, diethylene glycol monobutyl ether, tetrahydrofuran, 1,4-dioxane, ethyl acetate, butyl acetate, ethyl lactate, γ-butyrolactone, propylene glycol monomethyl ether acetate, propylene glycol monoethyl ether acetate, benzene, toluene, xylene, dimethylformamide, dimethylacetamide, and N-methylpyrrolidone.

Of these solvents, tetrahydrofuran, 1,4-dioxane, etc., may be used.

The thickness of the charge transport layer may be 3.0 μm or more and 50 μm or less, may be 5.0 μm or more and 40 μm or less, or may be 10 μm or more and 30 μm or less.

Furthermore, multiple solvents may be used in combination in order to adjust the drying rate of the coating solution for each layer and adjust the viscosity of the coating solution for each layer to a level suitable for coating.

### Process Cartridge and Electrophotographic Apparatus

The electrophotographic photosensitive member described thus far can be installed in a process cartridge that integrally supports at least one device selected from the group consisting of a charging device, a developing device, a transferring device, and a cleaning device. The process cartridge described above is characterized by being detachably attachable to an electrophotographic apparatus main body.

### Structure of Electrophotographic Apparatus

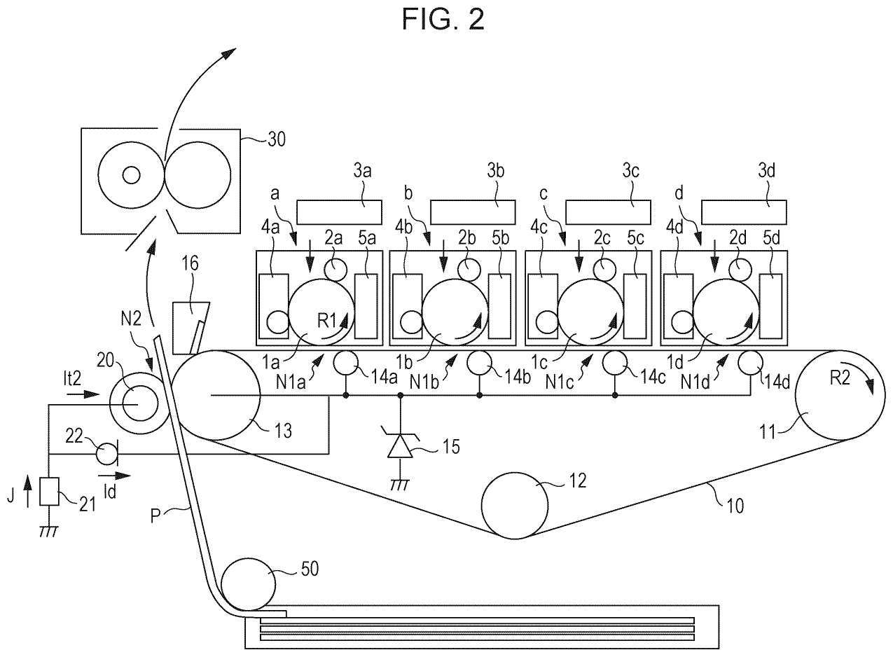

FIG. 2 illustrates an example of a schematic structure of an electrophotographic apparatus that includes process cartridges equipped with electrophotographic photosensitive members of the present disclosure.

Here, the electrophotographic apparatus of this embodiment is a so-called tandem type electrophotographic apparatus that has multiple image forming units a to d. A first image forming unit a, a second image forming unit b, a third image forming unit c, and a fourth image forming unit d respectively use a yellow (Y) toner, a magenta (M) toner, a cyan (C) toner, and a black (Bk) toner to form images. These four image forming units are spaced from each other and arranged in a line, and the structures of the image forming units are mostly substantially the same except for the color of toner housed therein. Thus, the electrophotographic apparatus of this embodiment is described by using the first image forming unit a as an example.

The first image forming unit a includes a photosensitive drum 1a which is a drum-shaped electrophotographic photosensitive member, a charging roller 2a which is a charging member, a developing device 4a, and a drum cleaning device 5a.

The photosensitive drum 1a is an image bearing member that bears a toner image and is rotatably driven at a predetermined circumferential velocity (process speed) in the arrow R¹ direction in the drawing. The developing device 4a houses a yellow toner and forms a yellow toner image on the photosensitive drum 1a. The drum cleaning device 5a is a device for recovering the toner adhering to the photosensitive drum 1a. The drum cleaning device 5a has a cleaning blade that comes into contact with the photosensitive drum 1a, and a waste toner box that houses, for example, the toner removed from the photosensitive drum 1a by the cleaning blade.

When a controller unit (not illustrated) such as a controller receives an image signal, an image forming operation starts, and the photosensitive drum 1a is rotatably driven. As the photosensitive drum 1a is rotated, the photosensitive drum 1a is uniformly charged to a predetermined voltage (charging bias) of a predetermined polarity (negative polarity in this embodiment) by the charging roller 2a and is exposed by an exposing device 3a according to the image signal. As a result, an electrostatic latent image corresponding to a yellow color component image of a target color image is formed on the photosensitive drum 1a. Next, the electrostatic latent image is developed by the developing device 4a at a development position and is visualized into a yellow toner image on the photosensitive drum 1a. Here, the normal charge polarity of the toner housed in the developing device 4a is negative, and the electrostatic latent image is reverse-developed by using a toner that has been charged by the charging roller 2a to have the same polarity as that of the photosensitive drum 1a. However, the technique of the present disclosure is not limited to the reversal development and can be applied to an electrophotographic apparatus that normally develops an electrostatic latent image with a toner charged to have a polarity opposite to the charge polarity of the photosensitive drum 1a.

An intermediate transfer belt 10 that can move endlessly has electroconductivity, forms a primary transfer part N1a by contacting the photosensitive drum 1a, and rotates at substantially the same circumferential velocity as that of the photosensitive drum 1a. The intermediate transfer belt 10 is stretched across an opposing roller 13 (opposing member), a drive roller 11 and a stretching roller 12 (stretching members), and a metal roller 14a, and is stretched at a total tensile force of 60 N by the stretching roller 12. The intermediate transfer belt 10 can move as the drive roller 11 is rotatably driven in the arrow R² direction in the drawing. In addition, the metal roller 14a and the opposing roller 13 are grounded via a Zener diode 15 serving as a constant voltage element.

The yellow toner image formed on the photosensitive drum 1a undergoes primary transfer from the photosensitive drum 1a to the intermediate transfer belt 10 as the yellow toner image passes through the primary transfer part N1a. The primary transfer residual toner remaining on the surface of the photosensitive drum 1a is cleaned and removed by the drum cleaning device 5a and then used in the image forming process downstream of the charging.

During the primary transfer, electric current is supplied to the conductive intermediate transfer belt 10 from a secondary transfer roller 20 that contacts the outer peripheral surface of the intermediate transfer belt 10 and that serves as a secondary transfer member. As the electric current supplied from the secondary transfer roller 20 flows in the circumferential direction of the intermediate transfer belt 10, the toner image undergoes primary transfer from the photosensitive drum 1a to the intermediate transfer belt 10. During this process, voltage J having a predetermined polarity (positive polarity in this embodiment) opposite of the normal charge polarity of the toner is applied from a transfer power supply 21 to the secondary transfer roller 20. In FIG. 2, the second, third, and fourth image forming units respectively have photosensitive drums 1b, 1c, and 1d, charging rollers 2b, 2c, and 2d, exposing devices 3b, 3c, and 3d, developing devices 4b, 4c, and 4d, drum cleaning devices 5b, 5c, and 5d, metal rollers 14b, 14c, and 14d, and primary transfer parts N1b, N1c, and N1d.

A second color magenta toner image, a third color cyan toner image, and a fourth color black toner image are formed in the same manner and are sequentially transferred and stacked on the intermediate transfer belt 10. As a result, toner images of four colors corresponding to a target color image are formed on the intermediate transfer belt 10. Subsequently, the toner images of four colors on the intermediate transfer belt 10 all together undergo secondary transfer onto a surface of a transfer medium P, such as a sheet of paper or an OHP sheet, fed from a sheet feeder 50 as the color images pass through a secondary transfer part N2 formed by the contact between a secondary transfer roller 20 and the intermediate transfer belt 10. The transfer medium P onto which the toner images of four colors have been transferred by secondary transfer is then heated and pressurized in a fixing device 30, as a result of which the toners of four colors melt, mix, and fix onto the transfer medium P. The toner remaining on the intermediate transfer belt 10 after the secondary transfer is cleaned and removed by a belt cleaning device 16 arranged to oppose the opposing roller 13 with the intermediate transfer belt 10 therebetween. Furthermore, a route that does not pass through the secondary transfer roller 20 and that electrically connects a transfer power supply 21 to each of the metal rollers 14 via a constant current diode 22 serving as a constant current element is provided. In addition, when voltage is applied from the transfer power supply 21 to the secondary transfer roller 20, a pinch-off current Id flows through the constant current diode 22 separate from electric current It2 flowing toward the secondary transfer part N2.

The electrophotographic photosensitive member of the present disclosure can be used in a laser beam printer, an LED printer, a copying machine, etc.

### EXAMPLES

The features of the present disclosure will now be described in further detail through examples and comparative examples. The following examples do not limit the present disclosure without departing from the gist thereof.

In the descriptions of the examples below, “parts” is on a mass basis unless otherwise noted. Furthermore, the thickness of each layer of the electrophotographic photosensitive members of Examples and Comparative Examples is was determined by using an Eddy current film thickness meter (product name: Fischerscope produced by Fischer Instruments K.K.) or by specific gravity conversion from the mass per unit area.

### Production Example of Electrophotographic Photosensitive Member 1

An alumite-treated aluminum tube was prepared as a conductive substrate. The length of the aluminum tube was 254 mm, the outer diameter was 30.0 mm, and the wall thickness t was 0.7 mm. The thickness of the alumite layer was 4.5 μm.

### Preparation of Photosensitive Layer-Forming Coating Solution

The following materials were prepared to obtain a mixed solvent:
Titanyl phthalocyanine, type IV: 45 parts by mass
Polyvinyl butyral: 27.5 parts by mass
Polymethylphenylsiloxane: 24.75 parts by mass
Polyhydroxystyrene: 2.75 parts by mass
2-Butanone: 2113.3 parts by mass
Cyclohexanone: 2113.3 parts by mass

A coating film of a charge generation layer-forming coating solution was formed on the aforementioned undercoat layer by dip-coating, and was heated and dried at a temperature of 100° C. for 15 minutes to form a charge generation layer having a thickness of 0.2 μm.

### Preparation of Charge Transport Layer-Forming Coating Solution

Charge transport material represented by formula (4) above: 1.47 parts by mass
Polycarbonate (trade name: Iupilon Z400 produced by Mitsubishi Engineering-Plastics Corporation): 14.7 parts by mass
Methyl palmitate (trade name: Palmitic Acid produced by Tokyo Chemical Industry Co., Ltd.): 1.47 parts by mass
Tetrahydrofuran: 50 parts by mass
1,4-Dioxane: 50 parts by mass

A coating film of a charge transport layer-forming coating solution was formed on the aforementioned charge generation layer by dip-coating, and was heated and dried at a temperature of 120° C. for 60 minutes to form a charge transport layer having a thickness of 18 μm.

### Electrophotographic Photosensitive Members 2 to 24

The process up to forming the photosensitive layer was performed as in Production example of electrophotographic photosensitive member 1 described above, but surface layer-forming coating solutions 2 to 24 used to form the charge transport layer were prepared by changing the materials as indicated in Table 1. Electrophotographic photosensitive members 2 to 24 were prepared by the same method as that for the electrophotographic photosensitive member 1 by using the prepared surface layer-forming coating solutions.

TABLE 1

| Charge | | | | | | | | | | | |
|---|---|---|---|---|---|---|---|---|---|---|---|
| transport | Charge transport | | | Binder | | | | | | | |
| layer- | material | | Methyl | resin | Particle | | | Solvent 1 | | Solvent 2 | |
| forming | | Parts | palmitate | Parts | | | Parts | | Parts | | Parts |
| coating | | by | Parts by | by | | Product | by | | by | | by |
| solution | Structure | mass | mass | mass | Type | name | mass | Type | mass | Type | mass |
| 1 | 1 | 1.47 | 1.47 | 14.7 | — | — | | THF | 50.0 | 1,4- | 50.0 |
| | | | | | | | | | | Dioxane | |
| 2 | 2 | 1.47 | 1.47 | 14.7 | — | — | | THF | 50.0 | 1,4- | 50.0 |
| | | | | | | | | | | Dioxane | |
| 3 | 3 | 1.47 | 1.47 | 14.7 | — | — | | THF | 50.0 | 1,4- | 50.0 |
| | | | | | | | | | | Dioxane | |
| 4 | 1 | 1.20 | 1.50 | 15.0 | — | — | | THF | 50.0 | 1,4- | 50.0 |
| | | | | | | | | | | Dioxane | |
| 5 | 1 | 3.78 | 1.26 | 12.6 | — | — | | THF | 50.0 | 1,4- | 50.0 |
| | | | | | | | | | | Dioxane | |
| 6 | 1 | 5.51 | 1.10 | 11.0 | — | — | | THF | 50.0 | 1,4- | 50.0 |
| | | | | | | | | | | Dioxane | |
| 7 | 1 | 6.23 | 1.04 | 10.4 | — | — | | THF | 50.0 | 1,4- | 50.0 |
| | | | | | | | | | | Dioxane | |
| 8 | 1 | 3.53 | 2.35 | 11.8 | — | — | | THF | 50.0 | 1,4- | 50.0 |
| | | | | | | | | | | Dioxane | |
| 9 | 1 | 3.92 | 0.654 | 13.1 | — | — | | THF | 50.0 | 1,4- | 50.0 |
| | | | | | | | | | | Dioxane | |
| 10 | 1 | 4.04 | 0.135 | 13.5 | — | — | | THF | 50.0 | 1,4- | 50.0 |
| | | | | | | | | | | Dioxane | |
| 11 | 1 | 3.36 | 1.12 | 11.2 | ITO | Indium | 11.2 | THF | 50.0 | 1,4- | 50.0 |
| | | | | | | tin oxide | | | | | |
| | | | | | | dispersion | | | | Dioxane | |
| | | | | | | (content: | | | | | |
| | | | | | | 30 wt %) | | | | | |
| 12 | 1 | 3.11 | 1.04 | 10.4 | ZnO | 23-K | 3.11 | THF | 50.0 | 1,4- | 50.0 |
| | | | | | | | | | | Dioxane | |
| 13 | 1 | 3.11 | 1.04 | 10.4 | TiO₂ | R25 | 3.11 | THF | 50.0 | 1,4- | 50.0 |
| | | | | | | | | | | Dioxane | |
| 14 | 1 | 3.11 | 1.04 | 10.4 | TiO₂ | MT-01 | 3.11 | THF | 50.0 | 1,4- | 50.0 |
| | | | | | | | | | | Dioxane | |
| 15 | 1 | 3.11 | 1.04 | 10.4 | TiO₂ | MT-500Z | 3.11 | THF | 50.0 | 1,4- | 50.0 |
| | | | | | | | | | | Dioxane | |
| 16 | 1 | 3.11 | 1.04 | 10.4 | TiO₂ | A190 | 3.11 | THF | 50.0 | 1,4- | 50.0 |
| | | | | | | | | | | Dioxane | |
| 17 | 1 | 4.33 | 1.44 | 14.4 | TiO | Titania | 43.3 | THF | 50.0 | 1,4- | 50.0 |
| | | | | | | nanoparticle | | | | Dioxane | |
| | | | | | | 1 (content: | | | | | |
| | | | | | | 10 wt %) | | | | | |
| 18 | 1 | 4.33 | 1.44 | 14.4 | TiO₂ | Titania | 43.3 | THF | 50.0 | 1,4- | 50.0 |
| | | | | | | nanoparticle | | | | Dioxane | |
| | | | | | | 2 (content: | | | | | |
| | | | | | | 10 wt %) | | | | | |
| 19 | 1 | 3.73 | 1.24 | 12.4 | ITO | Indium | 1.24 | THF | 50.0 | 1,4- | 50.0 |
| | | | | | | tin oxide | | | | Dioxane | |
| | | | | | | dispersion | | | | | |
| | | | | | | (content: | | | | | |
| | | | | | | 30 wt %) | | | | | |
| 20 | 1 | 3.70 | 1.23 | 12.3 | ITO | Indium | 2.06 | THF | 50.0 | 1,4- | 50.0 |
| | | | | | | tin oxide | | | | Dioxane | |
| | | | | | | dispersion | | | | | |
| | | | | | | (content: | | | | | |
| | | | | | | 30 wt %) | | | | | |
| 21 | 1 | 3.63 | 1.21 | 12.1 | ITO | Indium | 4.03 | THF | 50.0 | 1,4- | 50.0 |
| | | | | | | tin oxide | | | | Dioxane | |
| | | | | | | dispersion | | | | | |
| | | | | | | (content: | | | | | |
| | | | | | | 30 wt %) | | | | | |
| 22 | 1 | 3.24 | 1.08 | 10.8 | ITO | Indium | 14.4 | THF | 50.0 | 1,4- | 50.0 |
| | | | | | | tin oxide | | | | Dioxane | |
| | | | | | | dispersion | | | | | |
| | | | | | | (content: | | | | | |
| | | | | | | 30 wt %) | | | | | |
| 23 | 1 | 3.13 | 1.04 | 10.4 | ITO | Indium | 17.4 | THF | 50.0 | 1,4- | 50.0 |
| | | | | | | tin oxide | | | | Dioxane | |
| | | | | | | dispersion | | | | | |
| | | | | | | (content: | | | | | |
| | | | | | | 30 wt %) | | | | | |
| 24 | 1, 4, 5 | 3.36 | 1.12 | 11.2 | ITO | Indium | 11.2 | THF | 50.0 | 1,4- | 50.0 |
| | | | | | | tin oxide | | | | Dioxane | |
| | | | | | | dispersion | | | | | |
| | | | | | | (content: | | | | | |
| | | | | | | 30 wt %) | | | | | |

The materials used for preparing the charge transport layer-forming coating solutions were as follows.

### Charge Transport Material

For a charge transport material structure 1, a charge transport material represented by formula (4) was used.
For a charge transport material structure 2, a charge transport material represented by formula (6) was used.
For a charge transport material structure 3, a charge transport material represented by formula (7) was used.
For a charge transport material structure 4, a charge transport material represented by formula (3) was used.
For a charge transport material structure 5, a charge transport material represented by formula (5) was used.
The ratio in terms of parts by mass of the charge transport materials used in Example 24 was structure 1: structrue 4: structure 5=2:1:1.

### Particle

Indium tin oxide dispersion (primary particle diameter: less than 62 nm, content: 30 wt %, dispersion medium: isopropanol) (manufacturer: Sigma-Aldrich)
23-K (primary particle diameter: 185 nm) (manufacturer: Hakusui Tech Co., Ltd.)
R25 (primary particle diameter: 212 nm) (manufacturer: Sakai Chemical Industry Co., Ltd.)
MT-01 (primary particle diameter: 13 nm) (manufacturer: TAYCA Co., Ltd.)
MT-500Z (primary particle diameter: 36 nm) (manufacturer: TAYCA Co., Ltd.)
A190 (primary particle diameter: 151 nm) (manufacturer: Sakai Chemical Industry Co., Ltd.)
Titania nanoparticles (1) (primary particle diameter: 505 nm, content: 10 wt %, dispersion medium: ethanol) (manufacturer: Sigma-Aldrich)·
Titania nanoparticles (2) (primary particle diameter: 692 nm, content: 10 wt %, dispersion medium: water) (manufacturer: Sigma-Aldrich)

The average values obtained by observing the charge transport layer were indicated as the primary particle diameters of the particles.

### COMPARATIVE EXAMPLES

The process up to forming the charge transport layer was performed as in Production example of electrophotographic photosensitive member 1 described above, but surface layer-forming coating solutions c1 to c4 used to form the surface layer were prepared by changing the materials as indicated in Table 2. Electrophotographic photosensitive members C₁ to C₄ were prepared by the same method as that for the electrophotographic photosensitive member 1 by using the prepared surface layer-forming coating solutions.

TABLE 2

| Surface | Charge transport | | | | Binder | | | | | | | |
|---|---|---|---|---|---|---|---|---|---|---|---|---|
| layer- | material | | Additive | | resin | Particle | | | Solvent 1 | | Solvent 2 | |
| forming | | Parts | | Parts | Parts | | | Parts | | Parts | | Parts |
| coating | | by | | by | by | | Product | by | | by | | by |
| solution | Structure | mass | Type | mass | mass | Type | name | mass | Type | mass | Type | mass |
| c1 | 4 | 1.47 | Methyl | 1.47 | 14.7 | — | — | — | THF | 50.0 | 1,4- | 50.0 |
| | | | palmitate | | | | | | | | Dioxane | |
| c2 | 5 | 1.47 | Methyl | 1.47 | 14.7 | — | — | — | THF | 50.0 | 1,4- | 50.0 |
| | | | palmitate | | | | | | | | Dioxane | |
| c3 | 1 | 1.47 | Methyl | 1.47 | 14.7 | — | — | — | THF | 50.0 | 1,4- | 50.0 |
| | | | stearate | | | | | | | | Dioxane | |
| c4 | 1 | 1.47 | Methyl | 1.47 | 14.7 | — | — | — | THF | 50.0 | 1,4- | 50.0 |
| | | | myristate | | | | | | | | Dioxane | |

The materials used for preparing the charge transport layer-forming coating solutions were as follows.

### Charge Transport Material

For a charge transport material structure 6, a charge transport material represented by formula (8) below was used.
For a charge transport material structure 7, a charge transport material represented by formula (9) below was used.

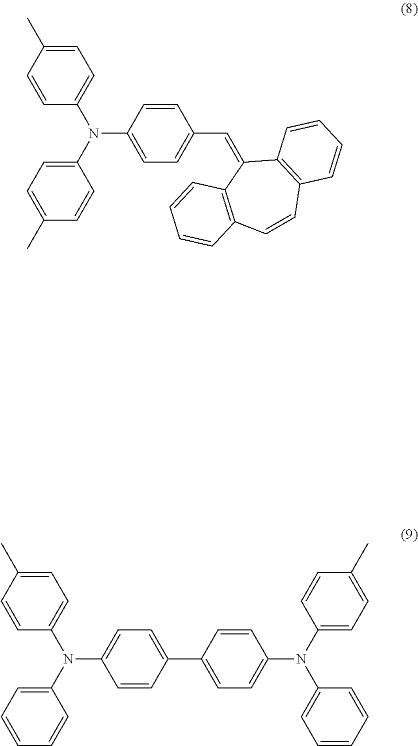 [SMILES: Cc1ccc(N(c2ccc(C)cc2)c2ccc(C=C3c4ccccc4C=Cc4ccccc43)cc2)cc1 ; Cc1ccc(N(c2ccccc2)c2ccc(-c3ccc(N(c4ccccc4)c4ccc(C)cc4)cc3)cc2)cc1]

### Additives

Methyl stearate (trade name: Stearic Acid produced by Tokyo Chemical Industry Co., Ltd.)
Methyl myristate (trade name: Myristic Acid produced by Tokyo Chemical Industry Co., Ltd.)

### Evaluation Procedure

### Deriving Mass of Each of Materials in Charge Transport Layer

The mass of each of the materials in the charge transport layer was calculated from the number of parts of each material used in the charge transport layer-forming coating solutions.

For example, a method described below is available to determine the mass from the electrophotographic photosensitive member.

Multiple pieces are detached from only the charge transport layer by using a cutting tool and are subjected to compositional analysis such as NMR, ESI-MS, or LC-CAD/MS/MSn to identify materials. Furthermore, polymerizable functional group-containing polymerizable monomers that constitute a composition, which will be polymerized into a binder resin contained in the charge transport layer, are identified.

Other pieces were subjected to thermogravimetric analysis such as TGA to measure the masses of the binder resin, the charge transport material, the additives, and the particles contained in the charge transport layer.

Sintered particles are subjected to compositional analysis such as SEM-EDS or XRF to identify the materials for the particles.

These techniques are employed to calculate the value of the ratio of the mass of the particle to the mass of the binder resin contained in the charge transport layer of the electrophotographic photosensitive member that has a charge transport layer containing the binder resin, the charge transport material, the additives, and the particle. Evaluation of number-average primary particle diameter of particle contained in charge transport layer

The values publicized by the manufacturers of the materials for the particles used can be referred for the number-average primary particle diameter of the particle contained in the charge transport layer. For example, a method described below is available for evaluation from the electrophotographic photosensitive member.

First, the entire electrophotographic photosensitive member was immersed in methyl ethyl ketone (MEK) in a graduated cylinder and irradiated with ultrasonic waves to peel off the resin layer, and then the substrate of the electrophotographic photosensitive member was taken out. Next, the insoluble matter (photosensitive layer and particle-containing charge transport layer) not soluble in MEK was filtered out and dried in a vacuum dryer. Furthermore, the obtained solid was suspended in a tetrahydrofuran (THF)/methylal (1:1 volume ratio) mixed solvent, and after filtering out the insoluble matter, the residue was collected and dried in a vacuum dryer. As a result of this operation, particles and resin of the charge transport layer were obtained. The residue was further heated to 500° C. in an electric furnace until the particles were the only solid matter, and the particles were then collected. In order to obtain the amount of particles required for measurement, multiple electrophotographic photosensitive members were processed in the same manner.

Some of the collected particles were dispersed in isopropanol (IPA), the resulting dispersion was added dropwise onto a grid mesh equipped with a support film (product name: Cu150J produced by JEOL Ltd.), and the particles were observed by STEM mode of a scanning transmission electron microscope (product name: JEM2800 produced by JEOL Ltd.). To facilitate counting of the particles, observation was performed at a magnification of 500,000× to 1,200,000×, and STEM images of 100 particles were taken. The particles were distinguished by using the EDX function of the scanning electron microscope. In doing so, the acceleration voltage was set to 20 kV, the probe size was set to 1 nm, and the image size was set to 1024×1024 pixel.

By using the obtained STEM images, the primary particle diameter was measured using image processing software “Image-Pro Plus (produced by Media Cybernetics Inc.)”.

First, select a scale bar displayed under the STEM image by using a straight line tool (Straight Line) in the tool bar. Under this condition, select Set Scale from the Analyze menu to open a new window in which the pixel distance of the selected straight line is input in the Distance in Pixels box. Input the value (for example, 100) of the scale bar in the Known Distance box of the window, input the unit (for example, nm) of the scale bar in the Unit of Measurement box, and click OK to complete the scale setting. Next, draw a straight line indicating the maximum diameter of the particle by using a straight line tool to calculate the particle diameter. The same operation was performed on 100 particles, and the number-average of the obtained values (maximum diameters) was assumed to be the primary particle diameter of the particle.

Another method for measuring the average primary particle diameter of the particle is as follows.

A 5 mm square sample piece was cut out from the electrophotographic photosensitive member by using a tool such as a saw. Here, the positions of cuts were a 38 mm position, 128 mm position, and a 218 mm position in the longitudinal direction, and a total of twelve 5 mm-square sample pieces were taken at these positions every 90° in the circumferential direction. A sample piece was set on a sample holder to allow observation of the charge transport layer. The sample holder with the sample piece set thereon was subjected to cross sectional observation with FIBSEM (trade name: NVision produced by ZEISS). The measurement conditions were the same as those described above.

In a cross sectional image of the charge transport layer obtained by FIBSEM, the particle diameters of the particles were measured. This operation was performed on all of the particles that existed within the cross sectional image and that had their all parts within the cross sectional image.

The arithmetic average of the obtained particle diameters of the particles was assumed to be the average particle diameter of that sample piece, and the arithmetic average of the average particle diameters of twelve sample pieces was assumed to be the average primary particle diameter of the particle contained in the charge transport layer.

### Image Smearing Evaluation

Image smearing of the electrophotographic photosensitive member was evaluated by using an electrophotographic apparatus (trade name: MS825dn produced by Lexmark International, Inc.) in an H/H environment (30.0° C., 80% RH). The image forming apparatus was modified so that the recording material feeding speed, the circumferential velocity of the intermediate transfer body, the circumferential velocity of the electrophotographic photosensitive member, and the voltage value during image forming could be modified.

The image forming apparatus, the toner cartridge used in the image forming apparatus, the electrophotographic photosensitive member, the letter-size recording material (trade name: XEROX Vitality produced by XEROX Corporation, grammage: 75 g/m²) were left to stand in an H/H environment for 24 hours. Subsequently, the electrophotographic photosensitive member was installed in the toner cartridge (cyan), and this toner cartridge only was attached to the image forming apparatus.

Next, the applied voltage was increased stepwise from −400 V in 100 V increments up to −2000 V, and the total current at each applied voltage was measured. A graph having a horizontal axis indicating the applied voltage and a vertical axis indicating the total current was plotted, an applied voltage at which the current value deviating from the linear approximation curve at an applied voltage of −400 V to −800 V was 100 μA was determined, and the applied voltage was set.

Next, a single-color solid image in cyan was output on a sheet of letter-size plain paper, and the image exposure amount was set such that the density on the paper was 1.45 as measured with a spectrodensitometer (trade name: X-Rite 504 produced by X-Rite, Incorporated).

Next, a letter-size square grid image having a line width of 0.1 mm and a line interval of 10 mm was output continuously on 180,000 sheets. After outputting the image, the main power of the electrophotographic apparatus was turned off, and the electrophotographic apparatus was left standing in an H/H environment for 3 days. Thereafter, the main power of the electrophotographic apparatus was turned on, a halftone image was immediately output on 10 sheets, the image smearing in the output images was visually observed, and the image smearing was evaluated according to the following standard. The image output on the 10th sheet was used as the evaluation result. The evaluation ratings down to D indicate practically acceptable levels. The evaluation results are indicated in Tables 3 and 4.

The image ranks were as follows:
Rank A: No abnormality was found in the halftone image.
Rank B: A slight decrease in density was observed in some part of the halftone image.
Rank C: A decrease in density was observed in some part of the halftone image.
Rank D: A slight decrease in density was observed in a wide range of the halftone image, and a larger decrease in density was observed in other part.
Rank E: A decrease in density was observed in a wide range of the halftone image.

### Endurance Evaluation

To evaluate the endurance of the electrophotographic photosensitive member surface layer, the thickness of the electrophotographic photosensitive member surface layer was measured before and after outputting 180,000 sheets for image smearing evaluation. The thickness after the image smearing evaluation was divided by the thickness before the image smearing evaluation, and the resulting value was used in evaluation. The smaller the amount of the surface layer scraped, the less likely the disadvantages caused by scraping would arise, and thus the rank is desirably A or close to the rank A. The evaluation results are indicated in Tables 3 and 4.

The endurance ranks were as follows:
Rank A: 1.0 to 0.9
Rank B: 0.8 to 0.7
Rank C: 0.6 or less

Here, changes in sensitivity were observed in Examples 4, 18, and 23, and bleeding of the additives was observed in Example 7.

TABLE 3

| | Type of electrophotographic | Image | Endurance |
|---|---|---|---|
| Example | photosensitive member | rank | rank |
| 1 | Electrophotographic | A | C |
| | photosensitive member 1 | | |
| 2 | Electrophotographic | A | C |
| | photosensitive member 2 | | |
| 3 | Electrophotographic | A | C |
| | photosensitive member 3 | | |
| 4 | Electrophotographic | A | C |
| | photosensitive member 4 | | |
| 5 | Electrophotographic | A | C |
| | photosensitive member 5 | | |
| 6 | Electrophotographic | A | C |
| | photosensitive member 6 | | |
| 7 | Electrophotographic | B | C |
| | photosensitive member 7 | | |
| 8 | Electrophotographic | A | C |
| | photosensitive member 8 | | |
| 9 | Electrophotographic | A | C |
| | photosensitive member 9 | | |
| 10 | Electrophotographic | A | C |
| | photosensitive member 10 | | |
| 11 | Electrophotographic | B | A |
| | photosensitive member 11 | | |
| 12 | Electrophotographic | C | A |
| | photosensitive member 12 | | |
| 13 | Electrophotographic | C | A |
| | photosensitive member 13 | | |
| 14 | Electrophotographic | C | A |
| | photosensitive member 14 | | |
| 15 | Electrophotographic | C | A |
| | photosensitive member 15 | | |
| 16 | Electrophotographic | C | A |
| | photosensitive member 16 | | |
| 17 | Electrophotographic | C | A |
| | photosensitive member 17 | | |
| 18 | Electrophotographic | D | A |
| | photosensitive member 18 | | |
| 19 | Electrophotographic | B | B |
| | photosensitive member 19 | | |
| 20 | Electrophotographic | B | B |
| | photosensitive member 20 | | |
| 21 | Electrophotographic | B | A |
| | photosensitive member 21 | | |
| 22 | Electrophotographic | B | A |
| | photosensitive member 22 | | |
| 23 | Electrophotographic | B | A |
| | photosensitive member 23 | | |
| 24 | Electrophotographic | B | A |
| | photosensitive member 24 | | |

TABLE 4

| Comparative | Type of electrophotographic | Image | Endurance |
|---|---|---|---|
| Example | photosensitive member | rank | rank |
| C1 | Electrophotographic | E | C |
| | photosensitive member C1 | | |
| C2 | Electrophotographic | E | C |
| | photosensitive member C2 | | |
| C3 | Electrophotographic | E | C |
| | photosensitive member C3 | | |
| C4 | Electrophotographic | E | C |
| | photosensitive member C4 | | |

The disclosure of this embodiment includes following features.

According to the present disclosure, in designing a charge transport layer that serves as a surface layer of an electrophotographic apparatus to contain a charge transport material having a structure resented by formula (1) above and methyl palmitate, an electrophotographic photosensitive member that can be used longer compared to the related art and can stably output high-quality images in long-term use at high temperature and high humidity can be provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-014459, filed Jan. 31, 2025, which is hereby incorporated by reference herein in its entirety.

What is claimed is:

1. An electrophotographic photosensitive member comprising:
a support;
a charge generation layer on the support; and
a charge transport layer on the charge generation layer, the charge transport layer constituting a surface layer,
wherein the charge transport layer includes a charge transport material, methyl palmitate, and a binder resin, and
the charge transport material includes a compound having a structure represented by formula (1):

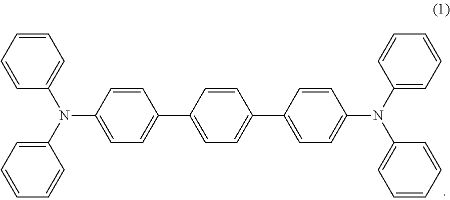 [SMILES: c1ccc(N(c2ccccc2)c2ccc(-c3ccc(-c4ccc(N(c5ccccc5)c5ccccc5)cc4)cc3)cc2)cc1]

2. The electrophotographic photosensitive member according to claim 1, wherein the compound having the structure represented by formula (1) is a compound represented by formula (2): 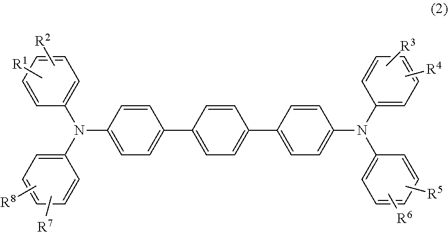
in formula (2), R¹ to R⁸ each independently represent a hydrogen atom, a substituted or unsubstituted alkyl group, a substituted or unsubstituted alkylene group, a substituted or unsubstituted phenyl group, or a group represented by formula (2A): 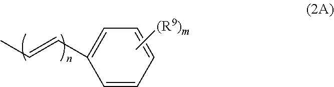
in formula (2A), n represents an integer of 1 or more and 5 or less, m represents an integer of 0 or more and 5 or less, and in a case that m represents an integer of 2 or more, each R⁹ independently represents an alkyl group having 1 or more and 4 or less carbon atoms or an alkoxy group having 1 or more and 4 or less carbon atoms.

3. The electrophotographic photosensitive member according to claim 2, wherein R¹ to R⁸ in formula (2) each independently represent an alkyl group having 1 or more and 3 or less carbon atoms.

4. The electrophotographic photosensitive member according to claim 1, wherein a value (MP/MB) of a ratio of a mass (MP) of the methyl palmitate to a mass (MB) of the binder resin in the charge transport layer is 0.1 or less.

5. The electrophotographic photosensitive member according to claim 1, wherein a value (MC/MB) of a ratio of a mass (MC) of the charge transport material to a mass (MB) of the binder resin in the charge transport layer is 0.1 or more and 0.5 or less.

6. The electrophotographic photosensitive member according to claim 1, wherein the charge transport layer includes a particle.

7. The electrophotographic photosensitive member according to claim 6, wherein the particle included in the charge transport layer is at least one selected from the group consisting of an indium tin oxide particle, a zinc oxide particle, and a titanium oxide particle.

8. The electrophotographic photosensitive member according to claim 6, wherein the particle has a number-average primary particle diameter of 10 nm or more and 500 nm or less.

9. The electrophotographic photosensitive member according to claim 6, wherein a value (MF/MB) of a ratio of a mass (MF) of the particle to a mass (MB) of the binder resin in the charge transport layer is 0.05 or more and 0.4 or less.

10. The electrophotographic photosensitive member according to claim 1, wherein the charge transport material includes at least one compound selected from the group consisting of a compound represented by formula (3), a compound represented by formula (4), and a compound represented by formula (5):

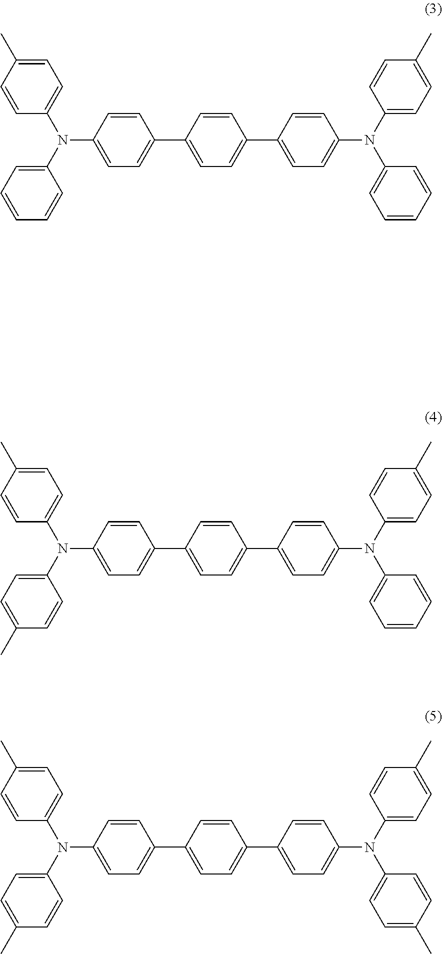 [SMILES: Cc1ccc(N(c2ccccc2)c2ccc(-c3ccc(-c4ccc(N(c5ccccc5)c5ccc(C)cc5)cc4)cc3)cc2)cc1 ; Cc1ccc(N(c2ccccc2)c2ccc(-c3ccc(-c4ccc(N(c5ccc(C)cc5)c5ccc(C)cc5)cc4)cc3)cc2)cc1 ; Cc1ccc(N(c2ccc(C)cc2)c2ccc(-c3ccc(-c4ccc(N(c5ccc(C)cc5)c5ccc(C)cc5)cc4)cc3)cc2)cc1]

11. The electrophotographic photosensitive member according to claim 6, wherein the particle in the charge transport layer is an indium tin oxide particle.

12. A process cartridge detachably attachable to a main body of an electrophotographic apparatus, the process cartridge integrally supporting and comprising:
an electrophotographic photosensitive member; and
at least one device selected from the group consisting of a charging device, a developing device, and a cleaning device,
wherein the electrophotographic photosensitive member includes a support, a charge generation layer on the support, and a charge transport layer on the charge generation layer,
the charge transport layer constitutes a surface layer of the electrophotographic photosensitive member,
the charge transport layer includes a charge transport material, methyl palmitate, and a binder resin, and
the charge transport material includes a compound having a structure represented by formula (1):

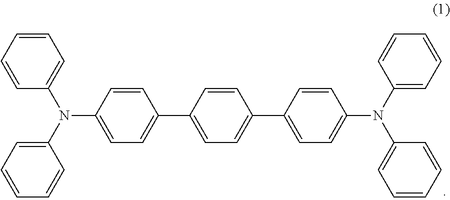 [SMILES: c1ccc(N(c2ccccc2)c2ccc(-c3ccc(-c4ccc(N(c5ccccc5)c5ccccc5)cc4)cc3)cc2)cc1]

13. An electrophotographic apparatus comprising:
an electrophotographic photosensitive member;
a charging device;
an exposing device;
a developing device; and
a transferring device,
wherein the electrophotographic photosensitive member includes a support, a charge generation layer on the support, and a charge transport layer on the charge generation layer,
the charge transport layer constitutes a surface layer of the electrophotographic photosensitive member,
the charge transport layer includes a charge transport material, methyl palmitate, and a binder resin, and
the charge transport material includes a compound having a structure represented by formula (1): 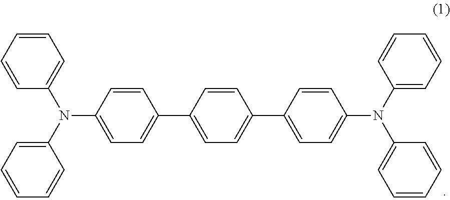 [SMILES: c1ccc(N(c2ccccc2)c2ccc(-c3ccc(-c4ccc(N(c5ccccc5)c5ccccc5)cc4)cc3)cc2)cc1]
